(19) United States
(12) Patent Application Publication
(10) Pub. No.: US 2006/0057119 A1
(43) Pub. Date: Mar. 16, 2006

(54) Biologic skin repair and enhancement

(76) Inventor: Peter K Law, Richmond Hill (CA)

Correspondence Address: HELLER EHRMAN WHITE & MCAULIFFE LLP, 1717 RHODE ISLAND AVE, NW, WASHINGTON, DC, 20036-3001, US

(21) Appl. No.: 10/525,361

(22) PCT Filed: Aug. 19, 2003
(86) PCT No.: PCT/US03/25896

§ 371 Date: Oct. 24, 2005

Related U.S. Application Data
(60) Provisional application No. 60/405,301, filed on Aug. 23, 2002.

(51) Int. Cl. A61K 35/36

(52) U.S. Cl. 424/93.7

(57) ABSTRACT

Muscle cell materials and methods are provided for refurbishing skin. Skin surfaces are prepared by removing dead cells and myoblasts are added in a cell-nutritive solution. An embodiment provides autologous human myoblast cells from the individual to be treated, serum from the individual, and an angiogenesis factor for stimulation of vascularization. Large 6 chondroitin sulfate may be used for controlled rapid cell fusion of the myoblasts. Foreskin fibroblast cell suspensions also may be used singly or in combination with myoblasts. In yet another embodiment non-immunogenic cells from another animal such as a pig with a double knockout mutation that affects foreign recognition may be added to cell surfaces.

This application claims priority to U.S. provisional application Ser. No. 60/405,301, filed Aug. 23, 2002, the entirety of which is hereby incorporated by reference.

### FIELD OF THE INVENTION

The invention relates generally to the repair of skin and more specifically to the use of living cells to repair and/or enhance skin.

### BACKGROUND OF THE INVENTION

Skin ages naturally with the passing of the years. Aging is accelerated by the action of the sun, which process is known as photo-aging and also by exposure to wind, salt and other factors. With aging, skin acquires or exhibits a variety of problems including lines, wrinkles, lack of firmness and elasticity, a rough, non-homogeneous stratum corneum, age spots and actinic keratosis.

Cosmetics that temporarily enhance the skin's appearance by masking irregularities with an opaque cosmetic film or coating have been used for thousands of years. Treatments that improve the quality and appearance of naked, unadorned skin rather than merely masking the irregularities also have been known or attempted since ancient times. Treatments employing fruit or milk acids, or vinegar rinses, if maintained consistently for long periods have probably succeeded in temporarily warding off some of the visual effects of aging. In recent years effective treatments are sought after for the rigors of aged skin and a large and growing market exists for products and services that are effective in providing structural improvements in the appearance of aged skin.

One class of products that provide structural improvements to the skin are skin renewal acids, notably alpha hydroxy acids and retinoic acids, or their esters. These acids, acidic compounds or esters work either intracellularly or intercellularly to stimulate skin cell proliferation. Some treatments employing skin renewal acids are disclosed in various patents to Yu and Van Scott, including, for example, U.S. Pat. Nos. 4,363,815, 5,091,171 and 5,422,370 and International Patent Publication No. WO 94/06640. Retinoids represent another class of skin-renewal stimulating products as disclosed for example in Kligman U.S. Pat. No. 5,051,449. However, these products are limited to altering small scale skin perturbations and do not work well on many people.

Skin renewal acids provide some benefits but tend to irritate, are slow to act and may cause dry skin problems. A particularly low irritation treatment employing a novel combination of lactic and salicylic acids is disclosed in international patent application publication No. WO/94/06640, which describes the topical application of a barrier disruption treatment effective to provide chronic and significant disruption of the skin's water barrier. The chronic disruption is maintained for a long enough period to induce structural improvements in skin. The disrupted water barrier is a natural impediment to the diffusion or evaporation of water vapor through the solid portion of the skin, and does not relate to sweating. Such techniques also generally are limited to small scale temporary improvements and are not applicable to many people.

### SUMMARY OF THE INVENTION

One embodiment of the invention is a method for refurbishing skin of an individual comprising removing dead cells from the surface of the skin to generate a prepared surface and applying myoblasts to the prepared skin surface in a myoblast cell-nutritive solution. Another embodiment of the invention is a myoblast cell suspension useful for skin enhancement of an individual, comprising autologous human myoblast cells from the individual, serum from the individual, an angiogenesis factor and large 6 chondroitin sulfate for controlled rapid cell fusion of the myoblasts. Another embodiment of the invention utilizes foreskin fibroblast cell suspension in a similar process, either singly or in combination with myoblasts. In yet another embodiment non-immunogenic cells from another animal such as a pig with a double knockout mutation that affects foreign recognition, such as a affecting alpha 1-3 galactose added to cell surfaces are used.

### DETAILED DESCRIPTION OF THE INVENTION

It was discovered that myoblasts cells from a cream like suspension could be applied as think layer(s) to a prepared skin surface such that the myoblasts survive, develop, and become integrated into the skin, and thereby fill in cracks and other crevices of the skin. The myoblasts smooth imperfections in the skin and provide further qualities such as resilientness, even coloration and strength.

In an advantageous embodiment the skin of an individual to be treated is first treated, preferably with a smooth abrasive or by one or more chemicals such as lactic acid. After treatment to remove dead cells, a suspension of myoblasts with or without foreskin fibroblasts, is smoothed into the desired area to fill blemishes, wrinkles, and/or holes. Then, warm moist oxygen-containing air, or more preferably pure oxygen is blown onto the treated area for at least 0.1, 1, 3, 6, 12, 24 or more hours. The area is left undisturbed for at least 12, 24 to 30 or more hours. The whole procedure may be repeated in intervals such as 1, 2, 3, 6 or 12 months to obtain smoother and younger looking skin.

In another embodiment the suspension comprises cells such as skin cells from another animal having a double knock out removal of a gene necessary for a foreign tissue specific antigen, such as alpha 1-3 galactose added to cell surfaces.

Myoblast Cells for the Treatment

Myoblasts can be autologous (obtained from the treated individual), obtained from other humans, or even obtained from other animals. If not autologous, cyclosporine preferably is administered in advance. Although other immune system suppressants may be used, cyclosporine for 5 days is preferred because it dampens inflammation in the skin by its effect on certain lymphocytes. Cyclosporine normally is taken by patients with severe skin disease for a minimum of several months and up to several years and typically is used at an oral dose of 5 mg/kg body weight per day.

If obtained from the individual undergoing treatment, one or more muscles preferably are stimulated with mechanical probing one, two to three, 4 to 5 or more days before removal. For example, four muscle sites may be stimulated: left and right deltoids and quadriceps using a 2.5 inch needle (26 gauge) 6 times per site after two hours of local anesthesia with Emgel. Two to three days later 0.5 gm of muscle may be biopsied using needle (punch) procedure for a total harvest of 2.0 gm. The cells are dissociated and grown into a culture of between 1 and 50 billion myoblasts.

Cultured myoblasts are suspended in any suitable medium. Preferably, particularly for autologous cells, the cells are resuspended in serum or blood obtained from the individual to be treated. For example, 50 to 100 ml of blood may be obtained from the individual and serum isolated. The desired amount of myoblasts are suspended preferably at about 100 million cells per ml in the serum (preferably at least 25%, 50% 75% or more serum), along with other factors such as, for example, NGF, insulin, VEGF165 (an angiogenesis factor) TGFbeta, angiotensin, dextrose, fetuin, lipid, albumin, large 6 chondroitin sulfate, and chick embryo extract (or other source of growth factors). A skilled artisan will appreciate how much of each factor to use. A routine optimization trial may use, for example, 0.01 ng to 1 ug/ml of angiogenesis factor. A more nutritive material such as dextrose, lipid, albumin, and chick embryo extract may be optimized at a higher concentration between 1 ug/ml to 25 mg/ml. Large 6 chondroitin sulfate may be used at a final concentration of between 0.01 ug/ml to 1 mg/ml and more preferably between 1 ug/ml and 100 ug/ml. The suspension medium is titrated to between 6.8 to 7.2 pH at room temperature. Cells are suspended and preferably administered shortly thereafter to apply to prepared skin. The cells should be at a concentration of between 1 million to 1 billion cells per milliliter, preferably, between 10 million to 500 million cells per milliliter and more preferably between 50 and 150 million cells per milliliter.

The cell suspension optionally includes one or more agents to adjust viscosity as is suited for mechanically adhering to a skin surface. For example, hyaluronic acid as described in U.S. Pat. No. 6,387,413 and porous tissue scaffoldings as described in U.S. Pat. No. 6,365,149 and foam composites as described in U.S. Pat. No. 6,306,424 may be used. In another embodiment that is useful for other conditions besides skin repair, such as blood vessel repair, and the repair of internal organs, myoblasts as described herein may be used to coat materials that are used as scaffolding with the body. Such materials may be treated to encourage myoblast adhesion, and in an embodiment may be coated with chondroitin sulfate. Plastics and inorganic materials useful for these further methods are known, for example as mentioned in U.S. Pat. No. 6,107,453; U.S. Pat. No. 5,843,781 and U.S. Pat. No. 5,503,771. In another embodiment, myoblast layers as described herein are used as coatings for artificial or biological organs, including solid tumors. Such myoblast layers may be used to both coat and protect as well as mechanically immobilize such organs, while allowing movement of large molecules and even of cells such as lymphocytes into and out of the immobilized organ.

In an embodiment particularly useful for filling in large skin areas such as large wrinkles, a mixture of individual myoblasts and small myotubes is used. The presence of myotubes helps mechanically bridge large gaps, and the ratio and average size of the myotubes in such mixtures may be prepared and adjusted as needed, as can be appreciated by a skilled artisan.

In another embodiment, foreskin fibroblasts may be used simply or in combination with myoblasts and or myotubes to produce similar results. Foreskin fibroblasts advantageously can provide smooth texture.

Preparation of Skin

Dead skin cells are removed prior to administration of myoblasts. An abrasive treatment may be used, such as massage with microgram water-tumbled quartz pebble and high grade body cream to remove the dead skin and debris. Other physical procedures can also be effective, for example, by stripping the skin with adhesive tape, or cyanoacrylate adhesive, or paraffin wax. Other disruption treatments may use chemical to remove barrier lipids from the stratum corneum. Organic solvents such as hexane, acetone or methanol and strong detergents such as sodium lauryl sulfate do not physically remove layers of the stratum corneum, but are effective in cleaning the surface, because they disrupt the water vapor barrier by removing significant lipid materials from the stratum corneum. An exfoliative can be used such as an alpha hydroxy acid, separately or in combination with an abrasive massage. After treatment, the treated skin should be rinsed with water, preferably at body temperature.

Application of Myoblasts to Prepared Skin

Preferably, inside a clean room, (class 100, class 1000, or class 10,000) myoblasts, optionally with myotubes and/or foreskin fibroblasts (or other cells) to fill in large expanses of skin are smoothed onto prepared surfaces to fill blemishes, wrinkles, and holes. Preferably the smoothed myoblast surfaces are exposed to oxygen and left alone for at least 6 hours, 12, 18 hours, 24 hours, 30 hours or even for at least 36 hours as desired. Preferably warm moist air that contains at least 20%, 35%, 50%, 70% or even above 95% oxygen is blown continuously onto the myoblast layer for at least 4, 6, 9, 12 or more hours. The treated area preferably is not cleaned for at least 24, 30, 36, 48 or more hours. In an embodiment an antibacterial reagent is added by spray or other procedure to the surface of the applied myoblasts, or may be added to the cell suspension before application to the skin surface.

Without wishing to be bound by any one theory for how embodiments of the invention work, it is pointed out that myoblasts can survive and grow in a wide range of culture, application solution and environmental conditions. Each such reagent is suitable for an embodiment of the invention. Much of the treated outer skin layer comprises fibroblasts, which are much larger than myoblasts. The applied myoblasts will develop into myotubes and can form a system of myofibers on the skin surface, allowing at least some fibroblasts to grow out and be shed from the skin surface. Furthermore, the myoblasts are mobile and can avoid hair follicles, thereby maintaining the hairy surface of skin. Still further, as the applied myoblasts (many of which never mature into muscle fibers) age and begin to die, they turn into connective tissue and can form some elastic or collagen. That is, some of the applied myoblast suspension degenerates into other tissue that forms a strong layer over the skin. Accordingly, one attribute of some embodiments of the invention is the removal or decrease in touch sensitivity. Another attribute is that treatments with myoblasts according to embodiments of the invention can produce an even (homogenous) desired skin color. In a particular embodiment, gene(s) tyrosinase and/or other enzymes involved in melanin reactions are turned on or regulated during in vitro culture of myoblasts, or immediately prior to applying myoblasts to the skin. This embodiment allows the use of myoblasts to correct for uneven pigmentation of skin.

Each document cited herein is specifically incorporated in its entirety by reference.

Of course, changes and modifications to the embodiments presented herein are readily understood by the skilled artisan after reading this specification and furthermore, such changes and modifications may be practiced within the scope of the appended claims.

1. A method for refurbishing skin of an individual comprising the steps:
a) removing dead cells from the surface of the skin to generate a prepared surface;
b) applying myoblasts to the prepared skin surface in a myoblast cell-nutritive solution.

2. The method of claim 1, wherein the myoblasts are autologous and obtained from muscle biopsy of the individual.

3. The method of claim 1, wherein the myoblasts are prepared from a human sample non-autologously.

4. The method of claim 1, wherein the myoblast cell-nutritive solution comprises serum, large 6 chondroitin sulfate and at least one angiogenic factor.

5. The method of claim 4, wherein the at least one angiogenic factor is VEGF165, TGFB, or angiotensin.

6. The method of claim 1, further comprising the step of circulating warm, moist oxygen onto the skin surface after application of myoblasts in a clean room.

7. The method of claim 1, wherein step a) is carried out by massage with micrograin water-tumbled quartz pebble and high grade body cream to remove dead skin and debris followed by rinsing.

8. The method of claim 1, wherein the myoblast cell-nutritive solution comprises serum obtained from the individual.

9. The method of claim 1, wherein at least 500 million myoblasts are applied in a concentration of at least 25 million cells per milliliter.

10. The method of claim 2, wherein the myoblasts obtained by needle biopsy after stimulation of one or more of the individual's muscles.

11. A myoblast cell suspension useful for skin enhancement of an individual, comprising autologous human myoblast cells from the individual, serum from the individual, an angiogenesis factor and large 6 chondroitin sulfate.

12. A myoblast cell suspension useful for skin enhancement of an individual as described in claim 11, further comprising NGF, insulin albumin and chick embryo extract.

13. A myoblast cell suspension useful for skin enhancement of an individual as described in claim 11, further comprising fetuin.

14. A myoblast cell suspension useful for skin enhancement of an individual, comprising myoblast cells, serum, and large 6 chondroitin sulfate in a cream suspension suitable for applying as a viscous mass to fill in surface imperfections of skin and to hide skin molds.

15. A myoblast cell suspension as described in claim 14, further comprising an angiogenic factor.

16. A foreskin fibroblast cell suspension useful for skin enhancement of an individual, comprising foreskin fibroblast cells, serum, and large 6 chondroitin sulfate in a cream suspension suitable for applying as a viscous mass to fill in surface imperfections of skin and to hide skin molds.
